(19) United States
(12) Patent Application Publication
(10) Pub. No.: US 2024/0390608 A1
(43) Pub. Date: Nov. 28, 2024

(54) COMBINATION SPIROMETER-INHALER

(71) Applicant: Willow Laboratories, Inc., Irvine (CA)

(72) Inventors: Kevin Hughes Pauley, Lake Forest, CA (US); Sai Kong Frank Lee, Irvine, CA (US)

(21) Appl. No.: 18/731,708

(22) Filed: Jun. 3, 2024

Related U.S. Application Data
(63) Continuation of application No. 17/355,752, filed on Jun. 23, 2021, now Pat. No. 12,029,844.
(60) Provisional application No. 63/044,269, filed on Jun. 25, 2020.
(60) Provisional application No. 63/071,310, filed on Aug. 27, 2020.

(51) Int. Cl. A61M 15/00

(52) U.S. Cl. CPC A61M 15/002; A61M 15/0018; A61M 15/0021; A61M 2205/3334; A61M 2205/3576; A61M 2205/583; A61M 2230/205; A61M 2230/42

(57) ABSTRACT

Various spirometer-inhaler systems and devices are disclosed. The spirometer-inhaler system may estimate physiological parameters of a user based on the user's inhaled and/or exhaled breaths received by the spirometer-inhaler device. The spirometer-inhaler device may comprise one or more flow paths. A first flow path may direct medication from a medicine canister to an opening of the device. A second flow path may direct a user's exhaled breath from the opening of the device to one or more flow measurement devices.

### INCORPORATION BY REFERENCE TO ANY PRIORITY APPLICATIONS

This application is a continuation of U.S. application Ser. No. 17/355,752 entitled “COMBINATION SPIROMETER-INHALER,” filed Jun. 23, 2021, which claims the benefit of priority to U.S. Provisional App. No. 63/044,269 entitled “COMBINATION SPIROMETER-INHALER,” filed Jun. 25, 2020 and U.S. Provisional App. No. 63/071,310 entitled “COMBINATION SPIROMETER-INHALER,” filed Aug. 27, 2020. Each of these applications are hereby incorporated by reference herein in their entireties. Any and all applications for which a foreign or domestic priority claim is identified in the Application Data Sheet as filed with the present application are hereby incorporated by reference and made a part of the present disclosure.

### FIELD OF THE DISCLOSURE

The general field of this disclosure is systems and devices related to managing a user's respiratory condition.

### BACKGROUND

Spirometers are often used to diagnose a variety of chronic lung conditions, such as asthma and chronic obstructive pulmonary disease (“COPD”), that may require the use of control and rescue inhalers. To complete a spirometer maneuver, many current spirometers require a user to attempt to use full tidal volume of their lungs and blow through a wide hole. Such a maneuver can result in coughing and general discomfort. Additionally, treatment of chronic lung conditions may require users carry around a number of items, including rescue medication, control medication, inhaler, spirometer, and a tracking device.

### SUMMARY

In some aspects of the disclosure, a spirometer-inhaler device for managing a user's respiratory condition is disclosed. A spirometer-inhaler device can include: a first portion that can removably receive a medicine canister; a second portion that can extend from the first portion and include a mouthpiece; a first flow path that can direct medication from the medicine canister to the opening of the mouthpiece; and a second flow path that can direct exhaled air from the opening of the mouthpiece to one or more flow rate measurement devices. The mouthpiece can include an opening and one or more propellers. The second flow path can include one of the one or more propellers.

In some aspects, the first portion can include a central cavity that can removably receive the medicine canister.

In some aspects, the first portion can optionally include a one way valve that can removably connect to the medicine canister and direct medication from the medicine canister to the opening of the mouthpiece.

In some aspects, the first flow path can include the one way valve and the opening of the mouthpiece.

In some aspects, the one way valve can form a seal with the medicine canister.

In some aspects, the seal can prevent the exhaled air from entering the first flow path.

In some aspects, the one way valve can deliver the medication from the medicine canister to the opening of the mouthpiece via the first flow path.

In some aspects, the first portion may not include a valve. For example, the first portion may include an interior gap that can direct medication from the medicine canister to the opening of the mouthpiece.

In some aspects, the spirometer-inhaler device can further include an interior flow pathway with a first end and a second end.

In some aspects, the second flow path can include the interior flow pathway and the opening of the mouthpiece.

In some aspects, the first end of the interior flow pathway can be located adjacent the opening of the mouthpiece. The second end of the interior flow pathway can be located on a side portion of the first portion.

In some aspects, the second flow path can further include at least one cover.

In some aspects, the at least one cover can include at least one shutter. The at least one shutter can include a lever portion and a cover portion that can cover the second end of the interior flow pathway.

In some aspects, the cover portion of the at least one shutter can be removed from the second end of the interior flow pathway in response to a user actuating the lever portion of the at least one shutter.

In some aspects, the at least one cover can include one or more of: a manual plug, a rotatable cover, a mechanical iris, or a cuspid one-way valve.

In some aspects, the interior flow pathway can include an interior tube.

In some aspects, the interior flow pathway can include a plurality of interior ribs. The plurality of interior ribs can guide the second flow path from the opening of the mouthpiece to the second end of the plurality of interior ribs.

In some aspects, the second end of the interior flow pathway can removably connect to the one or more flow rate measurement devices.

In some aspects, the spirometer-inhaler device can further include a display.

In some aspects, the display can be disposed on the first portion.

In some aspects, the spirometer-inhaler can further include at least one processor that can estimate one or more physiological parameters of the user based on measurements from the device.

In some aspects, the spirometer-inhaler device can further include a transmitter.

In some aspects, the transmitter can transmit the one or more physiological parameters of the user to one or more of: a network, a backend system, or one or more user devices.

In some aspects, the first flow path can include a propeller of the one or more propellers.

In some aspects, the propeller can be positioned adjacent the opening of the mouthpiece.

In some aspects, the propeller can spin in response to a negative flow rate within the mouthpiece.

In some aspects, the at least one processor can begin taking measurements in response to the propeller spinning.

In some aspects, the negative flow rate can be created within the mouthpiece by the user inhaling from the opening of the mouthpiece in use.

In some aspects, a propeller of the one or more propellers can be operatively connected to a turbine.

In some aspects of the disclosure, a system for delivering a medication to a user and measuring a plurality of physiological parameters of the user is disclosed. The system can include: a spirometer-inhaler device; a medicine canister that can deliver the medication to a first portion of the spirometer-inhaler device; and one or more measurement devices that can estimate one or more physiological parameters of the user. The spirometer-device can include: the first portion that can removably receive the medicine canister; a second portion that can extend from the first portion and include a mouthpiece that can include an opening and one or more propellers; a first flow path that can direct the medication from the medicine canister to the opening of the mouthpiece; and a second flow path that can direct exhaled air from the opening of the mouthpiece to one or more flow rate measurement devices. The second flow path can include one of the one or more propellers. The one or more measurement devices can include the one or more flow rate measurement devices.

In some aspects, the spirometer-inhaler device can further include a central hole that can removably receive the medicine canister.

In some aspects, the spirometer-inhaler device can further include a one way valve that can removably connect to the medicine canister and direct medication from the medicine canister to the opening of the mouthpiece.

In some aspects, the medicine canister can deliver the medication to the one way valve.

In some aspects, the first flow path can include the one way valve and the opening of the mouthpiece.

In some aspects, the central hole of the first portion can include an internal gap. The internal gap can direct medication from the medicine canister to the opening of the mouthpiece.

In some aspects, the spirometer-inhaler device can further include an interior flow pathway with a first end and a second end.

In some aspects, the first end of the interior flow pathway can be located adjacent the opening of the second portion and the second end of the interior flow pathway can be located on a side portion of the first portion.

In some aspects, the second flow path can include the interior flow pathway and the opening of the mouthpiece.

In some aspects, the second end of the interior flow pathway can be connectable to the one or more flow rate measurement devices.

In some aspects, the interior flow pathway can include one or more of: one or more interior tubes or one or more interior ribs.

In some aspects, the one or more flow rate measurement devices can estimate at least one of: forced inspiratory flow, force expiratory volume, forced expiratory flow, force vital capacity, maximal voluntary ventilation, inspiratory reserve volume, expiratory reserve volume, residual volume, functional residual capacity, total lung capacity, inspiratory capacity, tidal volume, vital capacity, maximal inspiratory pressure, or maximal expiratory pressure.

In some aspects, the one or more flow rate measurement devices can be connectable to the second flow path.

In some aspects, the one or more measurement devices can measure at least one of: oxygen saturation, respiratory rate, total hemoglobin level, level of carboxyhemoglobin, level of methemoglobin, pulse rate, or acoustic measurements.

In some aspects, the system can further include a display.

In some aspects, the display can be disposed on one or more of: the spirometer-inhaler device, the one or more measurement devices, or one or more user devices.

In some aspects, the system can further include a transmitter.

In some aspects, the transmitter can transmit the one or more physiological parameters of the user to one or more of: a network, a backend system, or one or more user devices.

### BRIEF DESCRIPTION OF THE DRAWINGS

The following drawings and the associated descriptions are provided to illustrate embodiments of the present disclosure and do not limit the scope of the claims.

FIG. 1 illustrates a block diagram of an example use environment for a spirometer-inhaler device.

FIGS. 2-3 illustrate perspective views of a spirometer-inhaler device.

FIG. 4 illustrates a close up view of a component of a spirometer-inhaler device.

FIG. 5 illustrates an exploded view the spirometer-inhaler device shown in FIGS. 2-3.

### DETAILED DESCRIPTION

Aspects of the disclosure will now be set forth in detail with respect to the figures and various examples. One of skill in the art will appreciate, however, that other configurations of the devices and methods disclosed herein will still fall within the scope of this disclosure even if not described in the same detail. Aspects of various configurations discussed do not limit the scope of the disclosure herein, which is instead defined by the claims following this description.

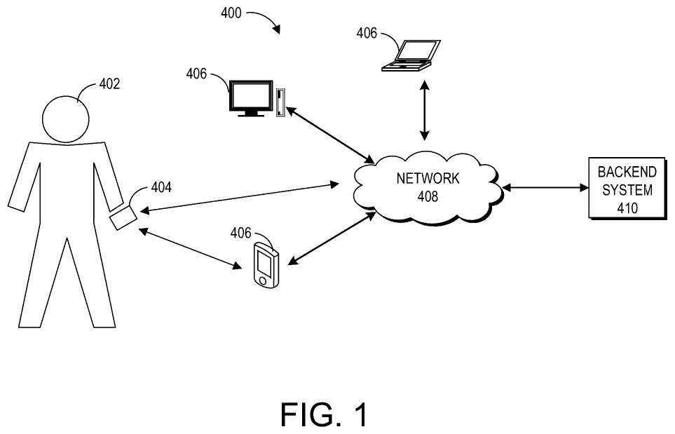

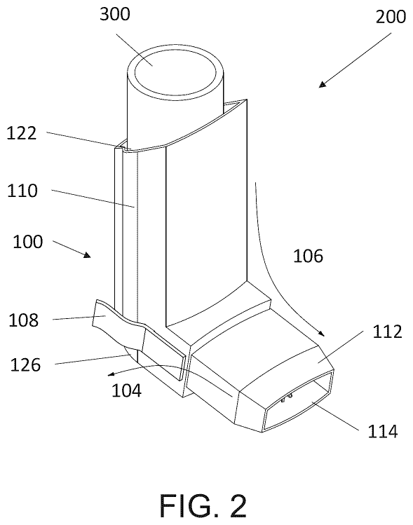

FIG. 1 illustrates an example use environment 400 that may make practical use of the health management systems, including a spirometer-inhaler device 100, described herein. For example, in the illustrated environment 400, one or more data collection devices 404 may collect data associated with a user 402. The data collection devices 404 may include the spirometer-inhaler device 100, as further described below with reference to FIGS. 2-4, one or more physiological sensors, one or more flow measurement devices, or other devices that may be used to monitor the health of the user. For example, the data collection devices 404 may include any device that may be used to monitor the respiratory health of the user. Example sensors may include optical heart rate sensors, electrical heart rate sensors accelerometers and gyroscopes, a GPS radio, temperature sensors, spectroscopic instruments, and/or other sensors. These example sensors may, for example, be used to take ECG readings, measure blood analytes, measure a user's movement, detect that a user is falling, or detect the user's position. Example devices may include measurement devices that measure one or more of: oxygen saturation, respiration rate, total hemoglobin levels, carboxyhemoglobin levels, methemoglobin levels, pulse-rate, pulse-rate variability, perfusion, Pleth variability index (PVI), pulmonary vascular resistance, and a variety of acoustic measurements. The spirometer-inhaler device 100 and the one or more data collection devices 404 may communicate with a backend system 410 through a network 408. Additionally or alternatively, the spirometer-inhaler device 100 and the one or more data collection devices 404 may communicate with one or more user devices 406. The user device 406 may include smart phones, tablet computers, laptop computers, smart watches, other mobile and wearable devices, desktop computers, networked televisions, networked game consoles, and/or the like. The device could also include a 3G/4G/5G modem for direct connection to the cloud to prevent the need for a smart phone. For example, the device 100 may measure a user's Forced Inspiratory Flow (FIF) value and display the user's FIF value on the user's smart phone or other electronic device. The one or more user devices 406 may communicate with the backend system 410 through a network 408.

The one or more user devices 406 can include a device associated with the user 402 of the one or more data collection devices 404, a device associated with a health care provider, a device associated with a third-party user, a device associated with a user's employer, the like or a combination thereof. For example, a user may measure the user's FIF value with the spirometer-inhaler device 100 and the spirometer-inhaler device 100 may transmit the user's estimated FIF value to the health care provider's device(s) so that the health care provider can monitor the user's respiratory health. Additionally, or alternatively, the device 100 can communicate the user's FIF value to the user's phone to display the user's FIF value on the phone's display and store the FIF value in the phone's memory so that the user can monitor the user's own respiratory health. For example, the user may download a mobile application associated with the spirometer-inhaler device 100 onto the user's phone or smartwatch. In order to access the user's respiratory health data, the user may access the mobile application and view the different physiological parameters being measured by the device 100 or the spirometer-inhaler system 200, as described below in reference to FIGS. 2-5. Moreover, in some embodiments, the device 100 can communicate the user's FIF value to the network 408. A user 402 and a health care provider may have respective login information to access the network 408 to monitor the user's 402 FIF values or other physiological parameters. In some examples, the one or more user devices 406 may upload, download, stream, display, analyze, read, write, access, or otherwise interact with data associated with the user 402, such as the data collected by the one or more data collection devices 404, data stored in the backend system 410 or other remote, non-local storage location, or data locally stored on the user devices 406. In some examples, the backend system 410 can include one or more hardware processors and/or storage systems capable of upload, download, display, analyze, read, write, access, or otherwise interact with the data or information associated with the user or other information associated with displaying and/or analyzing the data or information associated with the user.

The data collected by the spirometer-inhaler device 100, and the one or more data collection devices 404 can include a plurality of data associated with the user, including but not limited to health data. In some examples, health data can include heart rate, blood pressure, glucose, VO2 maximum, oxygen saturation, respiration rate, total hemoglobin levels, carboxyhemoglobin levels, methemoglobin levels, pulse-rate, pulmonary vascular resistance, symptoms and their severity, FIF, Force Expiratory Volume (FEV1), Forced Expiratory Flow (FEF), the like or a combination thereof. This collected data can be processed in a variety of different ways. For example, the spirometer-inhaler device 100 may include a processor that estimates a user's FIF value based on the user blowing into the device 100. The spirometer-inhaler device 100 may store the FIF data on a memory of the device 100 and display the FIF value on a display of the device 100. Additionally, or alternatively, the device 100 may send a measured value to a second device or the backend system 410, which processes the measured value to estimate a FIF value. The second device may display the estimated FIF value or the second device may send the FIF value to the device 100 so that the device 100 displays the FIF value.

FIGS. 2-5 illustrate different views of a spirometer-inhaler system 200 that can be used in the use environment 400 described in reference to FIG. 1. For example, the spirometer-inhaler system 200 may include a spirometer-inhaler device 100, a canister 300 configured to removably connect to the spirometer-inhaler device 100, one or more flow measurement devices (not shown) configured to removably connect to the spirometer-inhaler device 100, and/or one or more processors. The spirometer-inhaler system 200 may combine the assessment ability of a spirometer with the ability to deliver medication of an inhaler. Medication can include, for example, control medication or rescue medication associated with a respiratory disease, such as asthma.

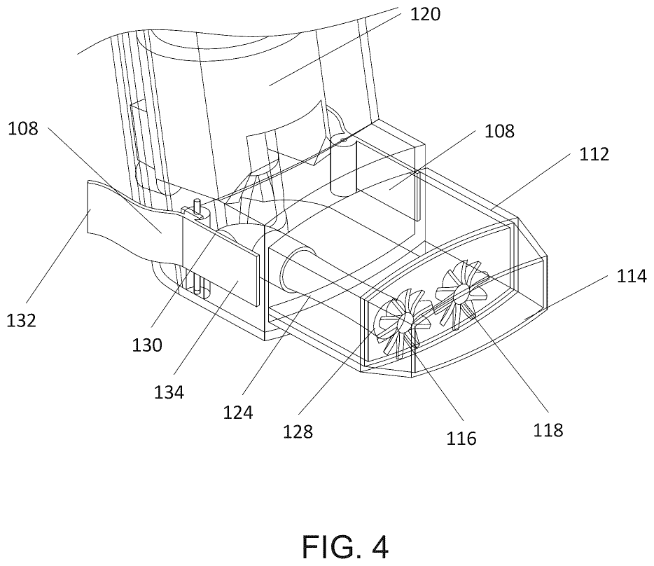

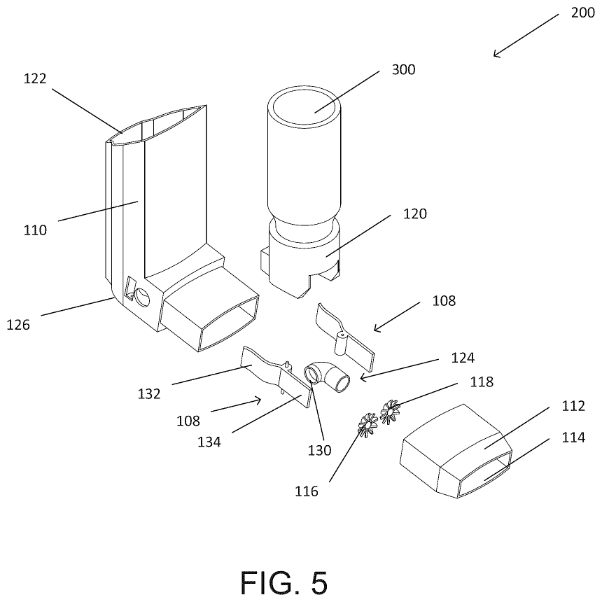

In some aspects, the spirometer-inhaler device 100 may include a first portion 110 and a second portion 112. In some embodiments, the device 100 may include a general L-shape, similar to a standard inhaler, with the first portion 110 forming the vertical component and the second portion 112 forming the horizontal component. Although the device 100 is shown as including a general L-shape, other shapes are contemplated. For example, the device may be cylindrical, conical, spherical, or have another shape in whole or in part. The first portion 110 may include a one-way valve 120, a first end 122 including a top opening, and a central cavity. The central cavity of the first portion 110 can extend from the first end 122 through the center of the first portion 110 and be configured to removably receive the canister 300. In some aspects, the first portion 110 itself can be one of the two (or more) channels required for flow estimations. The first portion 110 can have a one-way valve 120 (such as illustrated in FIGS. 4 and 5) to improve the simplicity of the device 100. In some aspects, the one-way valve 120 may be configured to removably connect with the canister 300. For example, the one-way valve 120 may include a threaded recess configured to engage with a threading on an end of the canister 300 such that the end of the canister 300 can be sealed to the one-way valve 120 while a body portion of the canister 300 can be housed within the central cavity of the first portion 110. Additionally or alternatively, the canister 300 may removably connect to the one-way valve 120 with a snap-fit engagement, detent, or other coupling mechanism. The canister 300 may form a seal with the one-way valve 120 so as to prevent a user's expiratory flow from traveling via a first flow path 106 of the system 200, as described below. The one-way valve 120 may be configured to allow fluid to flow from the canister 300 to an opening 114 of the second portion 112 and prevent fluid from flowing in the opposite direction. In some examples, the second portion 112 can include at least a portion of the first flow path 106. In addition to, or alternative to, the one-way valve 120, the first portion 110 can include an internal gap or aperture within the central cavity of the first portion 110 adapted to allow fluid to flow from the canister 300 to the opening 114 of the second portion 112 and prevent fluid from flowing in the opposite direction.

In some aspects, the second portion 112 of the device 100 may extend from the first portion 110, adjacent a second end 126 of the first portion 110 opposite the first end 122. The second portion 112 may include a mouth piece 112 with an opening 114 and at least one propeller 116, 118. In some embodiments, the system 200 may include a turbine with a turning wheel. In some embodiments, the system 200 includes the canister 300. The canister 300 may be a medicine canister that holds control medication, rescue medication, or the like.

In some configurations, the disclosed system 200 can include two or more flow paths 104, 106. A first flow path 106 of the two or more flow paths may include the canister 300, the one-way valve 120 of the first portion 110, and/or the second portion 112. The direction of the first flow path 106 is indicated by arrow 106 in FIG. 2. The one-way valve 120 can be configured to direct medication from the canister 300 along the first flow path 106 to the user during inhalation while preventing the user's exhaled breaths from traveling along the first flow path 106. The second portion 112 may optionally include a first propeller 118 positioned in the first flow path 106 adjacent to the opening 114 of the second portion 112. The first propeller 118 may be configured to better deliver the medication into the user's lungs. For example, when a user places the mouthpiece 112 in the user's mouth and inhales to administer the medication, a negative flow rate is created that may cause the first propeller 118 to spin and propel the medication from the canister 300 into the user's mouth. Additionally or alternatively, the first propeller 118 spinning can notify the one or more processors of the system 200 that the medication is being administered and the one or more processors of the system 200 can detect how much of the medication was administered and the depth of the user's breath. For example, the one or more processors of the system 200 may be configured to measure the amount of medication administered and the depth of the user's inhaled breath when the first propeller 118 begins spinning. The measured depth of the user's inhaled breath can be used as another variable to estimate various physiological parameters of the user.

In some configurations, the spirometer-inhaler system 200 can take additional measurements to enable better predictive capacity of FEV1 or FEF. For example, the system 200 can utilize the patient's inhalation to better estimate common respiratory measurements. Moreover, the system 200 can use additional measurements, such as oxygen saturation, respiration rate, and the like, to improve the estimation of all variable types listed in the present disclosure. For example, these additional measurements can be utilized as spot-check, trended, or modeled forms of these data types.

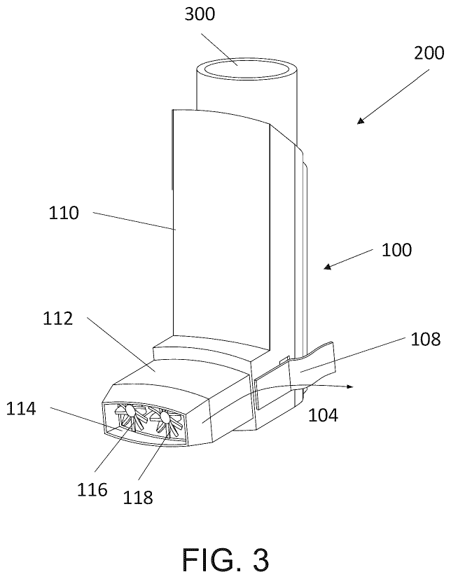

In some configurations, the system 200 includes a second flow path 104 that may include portions of the second portion 120. The direction of the second flow path 104 is indicated by arrow 104 in FIGS. 2 and 3. The second portion 120 may further include an interior flow pathway 124 and a second propeller 116. The system 200 is configured to direct the user's exhaled breaths along the second flow path 104. For example, referring to FIGS. 4 and 5, a user may exhale into the opening 114 and the user's exhaled breath is directed through the interior flow pathway 124. For example, the interior flow pathway 124 can comprise one or more tubes or one or more ribs adapted to guide the second flow path 104. A first end 128 of the interior flow pathway 124 can be positioned adjacent the opening 114 and the second propeller 116. A second end 130 of the interior flow pathway 124 can be positioned on a side portion of the first portion 110, adjacent the second end 126 of the first portion 110. Although the first and second ends 128, 130 of the interior flow pathway 124 are illustrated in this manner, the first and second ends 128, 130 may be located anywhere on the device 100 that allows for at least two flow paths 104, 106.

In some aspects, the second end 130 of the interior flow pathway 124 is configured to be removably connected to the one or more flow measurement devices. The one or more flow measurement devices can include devices that measure FIF values, FEV1 values or FEF values. When the one or more flow measurement devices are connected to the interior flow pathway 124, the second flow path 104 may include the one of the one or more flow measurement devices.

The interior flow pathway 124 may include a hole, which extends from the first end 128 of the interior flow pathway 124 to the second end 130 of the interior flow pathway 124, that may be smaller than the wider standard holes of current spirometers. For example, the hole of the interior flow pathway 124 can be approximately half to a quarter of the size of the wider standard holes of current spirometers.

As described above, the system 200 can comprise one or more processors. The processor(s) may be located within the spirometer-inhaler device 100, the one or more flow measurement devices, and/or the backend system 410. In some embodiments, the processor(s) can be configured to extrapolate the measurements from the smaller hole of the interior flow pathway 124 to assess the air flow of the wider standard holes of current spirometers. The smaller hole enables the prediction of FIF, FEV1 or FEF without requiring the user to completely expel the air (or completely deplete the air) from their lungs. Advantageously, reducing the amount of lung depletion may improve user comfort. In some configurations, the processor may also be configured to upload, download, display, analyze, read, write, access, or otherwise interact with the data or information associated with the user, such as data collected by the one or more data collection device 404 including the device 100 and/or the one or more flow measurement devices, or other information associated with displaying and/or analyzing the data or information associated with the user.

Additionally or alternatively, the one or more processors of the system 200 may estimate biometric parameters other than FIF, FEV1, or FEF, including, but not limited to, Force Vital Capacity (FVC), Maximal Voluntary Ventilation (MVV), Inspiratory Reserve Volume (IRV), Expiratory Reserve Volume (ERV), Residual Volume (RV), Functional Residual Capacity (FRC), Total Lung Capacity (TLC), IC (Inspiratory Capacity), tidal volume, vital capacity, and Maximal Inspiratory Pressure (MIP), and Maximal Expiratory Pressure (MEP). To effectively estimate some of these other values, the user may have to deplete their lungs.

In some aspects, the second flow path 104 may include a turbine with a first end and a second end. The first end of the turbine can include a turning wheel that can be adapted to cause the airflow that passes through the turning wheel to rotate helically. The turbine can also include a vane between the turning wheel and the second end of the turbine. The helically rotating airflow can cause the internal vane to rotate. The rotations of the internal vane can be detected by a sensor which can send the sensor signals to the one or more processors. The processor(s) can determine certain physiological parameters related to a patient's lung functionality. For example, the processor(s) can determine a user's FIF, FEV1 and/or FEF values.

In some embodiments, the system 200 may comprise a memory configured to store user's instructions. For example, one or more processors of the system 200 may access the user's instructions from the memory and display the instructions on a display of the system 200. The user's instructions may instruct the user on how to measure different physiological parameters with the system 200. For example, if the user wants to measure the user's FVC value, the user's instructions may include instructing the user to deplete the user's lungs into the device 100. Additionally or alternatively, the user can access the user's instructions on the user's phone via the mobile application associated with the spirometer-inhaler device 100. In some aspects, the user can access the user's instructions on any of the user devices 406 via an application, a website, or the like.

In some configurations, the spirometer-inhaler system 200 may further include a display (not shown) configured to display relevant information, one or more sensors, a battery, and a transmitter. In some aspects, the display can be positioned on the spirometer-inhaler device 100 and be electronically connected to the battery and the one or more processors. Additionally or alternatively, the display may be positioned on the one or more user devices 406 and/or the one or more flow measurement devices. For example, when a user blows into the device 100, the display can show the estimated FIF value. In some embodiments, the device 100 may include one or more sensors that can be electronically connected to the one or more processors and the battery. Additionally or alternatively, the one or more sensors may be located within the one or more flow measurement devices. The one or more sensors may include one or more differential pressure sensors.

In some configurations, the transmitter that can be electronically connected to the battery and the one or more processors. Additionally or alternatively, the transmitter may be located within the one or more flow measurement devices. In some aspects, the transmitter may be configured to transmit the health data from the device 100 or the one or more flow measurement devices to the network 408, the back end system 410, and/or the one or more user devices 406.

In some embodiments, one or more covers 108 may be incorporated in the second flow path 104 to enable switching between the one or more flow measurement devices. For example, the one or more covers 108 can include one or more shutters 108 with a lever portion 132 connected to a cover portion 134 configured to cover the second end 130 of the interior flow pathway 124. The user can push on the lever portion 132 removing the cover portion 134 from the second end 130 of the interior flow pathway 124, thereby opening the second flow path 104. With the second flow path 104 open, the user can removably connect an external device to the second flow path 104. The external device, for example, can include the one or more flow measurement devices. Additionally, or alternatively, the one or more covers 108 may include a manual plug, a cover adapted to open and close via a rotation mechanism, a mechanical iris, a cuspid one-way valve, a flap, or the like.

### Terminology

Conditional language used herein, such as, among others, “can,” “could,” “might,” “may,” “e.g.,” and the like, unless specifically stated otherwise, or otherwise understood within the context as used, is generally intended to convey that certain, certain features, elements and/or steps are optional. Thus, such conditional language is not generally intended to imply that features, elements and/or steps are in any way required or that one or more implementations necessarily include logic for deciding, with or without other input or prompting, whether these features, elements and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain implementations require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain implementations, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication.

The methods and tasks described herein may be performed and fully automated by a computer system. The computer system may, in some cases, include multiple distinct computers or computing devices (for example, physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (for example, solid state storage devices, disk drives, etc.). The various functions disclosed herein may be embodied in such program instructions, and/or may be implemented in application-specific circuitry (for example, ASICs or FPGAs) of the computer system. Where the computer system includes multiple computing devices, these devices may, but need not, be co-located. The results of the disclosed methods and tasks may be persistently stored by transforming physical storage devices, such as solid state memory chips and/or magnetic disks, into a different state. The computer system may be a cloud-based computing system whose processing resources are shared by multiple distinct business entities or other users.

Depending on the implementation, certain acts, events, or functions of any of the processes or algorithms described herein can be performed in a different sequence, can be added, merged, or left out altogether (for example, not all described operations or events are necessary for the practice of the algorithm). Moreover, in certain implementations, operations or events can be performed concurrently, for example, through multi-threaded processing, interrupt processing, or multiple processors or processor cores or on other parallel architectures, rather than sequentially.

The various illustrative logical blocks, modules, routines, and algorithm steps described in connection with the disclosure herein can be implemented as electronic hardware (for example, ASICs or FPGA devices), computer software that runs on general purpose computer hardware, or combinations of both. Various illustrative components, blocks, and steps have been described above generally in terms of their functionality. Whether such functionality is implemented as specialized hardware versus software running on general-purpose hardware depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the disclosure herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. A processor device can include an FPGA or other programmable device that performs logic operations without processing computer-executable instructions. A processor device can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the disclosure herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

While the above detailed description has shown, described, and pointed out novel features, it can be understood that various omissions, substitutions, and changes in the form and details of the devices or algorithms illustrated can be made without departing from the spirit of the disclosure. As can be recognized, certain portions of the description herein can be embodied within a form that does not provide all of the features and benefits set forth herein, as some features can be used or practiced separately from others. The scope of certain implementations disclosed herein is indicated by the appended claims rather than by the foregoing description. All changes which come within the meaning and range of equivalency of the claims are to be embraced within their scope.

1. (canceled)

2. A spirometer-inhaler device comprising:
a first portion configured to removably receive a medicine canister;
a second portion extending from the first portion, the second portion comprising a mouthpiece, wherein the mouthpiece comprises an opening and a plurality of flow rate sensors;
a first flow path configured to direct medication from the medicine canister to the opening of the mouthpiece, wherein the first flow path comprises a first flow rate sensor configured to measure flow rate of inhaled air; and
a second flow path configured to direct exhaled air from the opening of the mouthpiece to one or more flow rate measurement devices, wherein the second flow path comprises a second flow rate sensor, the second flow rate sensor configured to measure flow rate of exhaled air.

3. The spirometer-inhaler device of claim 2, wherein the first flow rate sensor comprises a first propeller.

4. The spirometer-inhaler device of claim 3, wherein the first propeller is configured to spin in response to a negative flow rate within the mouthpiece.

5. The spirometer-inhaler device of claim 4, wherein the negative flow rate is created within the mouthpiece by a user inhaling from the opening of the mouthpiece during use.

6. The spirometer-inhaler device of claim 2, wherein the second flow rate sensor comprises a second propeller.

7. The spirometer-inhaler device of claim 2, wherein the first portion comprises a central cavity configured to removably receive the medicine canister.

8. The spirometer-inhaler device of claim 2, wherein the first portion comprises a one way valve configured to removably connect to the medicine canister and to direct medication from the medicine canister to the opening of the mouthpiece.

9. The spirometer-inhaler device of claim 8, wherein the first flow path comprises the one way valve and the opening of the mouthpiece.

10. The spirometer-inhaler device of claim 9, wherein the one way valve is configured to form a seal with the medicine canister, the seal configured to prevent exhaled air from entering the first flow path.

11. The spirometer-inhaler device of claim 9, wherein the one way valve is configured to deliver the medication from the medicine canister to the opening of the mouthpiece via the first flow path.

12. The spirometer-inhaler device of claim 2, wherein the first portion comprises an internal gap configured to direct medication from the medicine canister to the opening of the mouthpiece.

13. The spirometer-inhaler device of claim 2, further comprising an interior flow pathway with a first end and a second end, wherein the second flow pathway comprises the interior flow pathway and the opening of the mouthpiece.

14. The spirometer-inhaler device of claim 13, wherein the first end of the interior flow pathway is located adjacent the opening of the mouthpiece, wherein the second end of the interior flow pathway is located on a side portion of the first portion.

15. The spirometer-inhaler device of claim 14, wherein the second flow path further comprises at least one cover.

16. The spirometer-inhaler device of claim 15, wherein the at least one cover comprises at least one shutter comprising a lever portion and a cover portion configured to cover the second end of the interior flow pathway.

17. The spirometer-inhaler device of claim 13, wherein the interior flow pathway comprises a plurality of interior ribs configured to guide the second flow path from the opening of the mouthpiece to the second end of the plurality of interior ribs.

18. The spirometer-inhaler device of claim 13, wherein the second end of the interior flow pathway is configured to removably connect to the one or more flow rate measurement devices.

19. The spirometer-inhaler device of claim 2, further comprising at least one processor configured to estimate one or more physiological parameters of a user based on measurements from the device.

20. The spirometer-inhaler device of claim 2, wherein the first flow rate sensor is positioned adjacent the opening of the mouthpiece.

21. The spirometer-inhaler device of claim 2, comprising a processor configured to begin taking measurements in response to the flow rate sensor detecting air flow.
